(19) United States
(12) Patent Application Publication
(10) Pub. No.: US 2023/0290020 A1
(43) Pub. Date: Sep. 14, 2023

(54) IMAGE PROCESSING APPARATUS, IMAGE PROCESSING METHOD, AND STORAGE MEDIUM

(71) Applicants: CANON KABUSHIKI KAISHA, Tokyo (JP); CANON MEDICAL SYSTEMS CORPORATION, Tochigi (JP)

(72) Inventor: Toru TANAKA, Chiba (JP)

(21) Appl. No.: 18/180,165

(22) Filed: Mar. 8, 2023

(30) Foreign Application Priority Data
Mar. 10, 2022 (JP) 2022-037244

(51) Int. Cl. G06T 11/00

(52) U.S. Cl. CPC G06T 11/005; G06T 2200/04; G06T 2210/41

(57) ABSTRACT

An image processing apparatus comprises: a region obtaining unit configured to obtain a first region in a tomographic image included in a three-dimensional image, and a second region different from the first region; a setting unit configured to set a first slab having a first slab thickness for the first region, and a second slab having a second slab thickness, which is different from the first slab thickness, for the second region; and a generation unit configured to generate a projection image regarding the tomographic image using the first slab and the second slab.

### BACKGROUND OF THE INVENTION

### Field of the Invention

The present invention relates to an image processing apparatus, an image processing method, and a storage medium.

### Description of the Related Art

In the medical field, there is a technique of improving the visibility of the structure of a site by presenting to a user a projection image of a three-dimensional image obtained by capturing by various modalities.

Japanese Patent Laid-Open No. 2009-273644 discloses a technique of improving the visibility of the structure of a site by displaying a projection image obtained by Maximum Intensity Projection (MIP) of a section in a three-dimensional image at a predetermined thickness (slab thickness).

### SUMMARY OF THE INVENTION

The visibility of a plurality of sites is poor in a projection image obtained by projecting at the same slab thickness an entire section on which the sites are captured.

The present invention has been made to overcome the above-described drawbacks, and provides an image processing technique capable of improving the visibility of a projection image in a three-dimensional image.

According to one aspect of the present invention, there is provided an image processing apparatus comprising: a region obtaining unit configured to obtain a first region in a tomographic image included in a three-dimensional image, and a second region different from the first region; a setting unit configured to set a first slab having a first slab thickness for the first region, and a second slab having a second slab thickness, which is different from the first slab thickness, for the second region; and a generation unit configured to generate a projection image regarding the tomographic image using the first slab and the second slab.

According to another aspect of the present invention, there is provided an image processing apparatus comprising: a region obtaining unit configured to obtain a first region including one of a lung and a heart in a tomographic image included in a three-dimensional image, and a second region different from the first region; a setting unit configured to set a first slab having a first slab thickness for the first region, and a second slab having a second slab thickness, which is different from the first slab thickness, for the second region; and a generation unit configured to generate a projection image regarding the tomographic image using the first slab and the second slab.

According to still another aspect of the present invention, there is provided an image processing method for an image processing apparatus, comprising: obtaining a first region in a tomographic image included in a three-dimensional image, and a second region different from the first region; setting a first slab having a first slab thickness for the first region, and a second slab having a second slab thickness, which is different from the first slab thickness, for the second region; and generating a projection image regarding the tomographic image using the first slab and the second slab.

According to yet another aspect of the present invention, there is provided an image processing method for an image processing apparatus, comprising: obtaining a first region including one of a lung and a heart in a tomographic image included in a three-dimensional image, and a second region different from the first region; setting a first slab having a first slab thickness for the first region, and a second slab having a second slab thickness, which is different from the first slab thickness, for the second region; and generating a projection image regarding the tomographic image using the first slab and the second slab.

Further features of the present invention will become apparent from the following description of exemplary embodiments (with reference to the attached drawings).

### BRIEF DESCRIPTION OF THE DRAWINGS

FIG. 1 is a block diagram showing the arrangement of an image processing system including an image processing apparatus according to the first embodiment;

FIG. 2 is a block diagram showing a functional arrangement of the control unit of the image processing apparatus in the first embodiment;

FIG. 3 is a flowchart showing an example of an overall processing procedure in the first embodiment; and

FIG. 4 is a view for schematically explaining projection processing.

### DESCRIPTION OF THE EMBODIMENTS

Hereinafter, embodiments will be described in detail with reference to the attached drawings. Note, the following embodiments are not intended to limit the scope of the claimed invention. Multiple features are described in the embodiments, but limitation is not made to an invention that requires all such features, and multiple such features may be combined as appropriate. Furthermore, in the attached drawings, the same reference numerals are given to the same or similar configurations, and redundant description thereof is omitted.

### First Embodiment

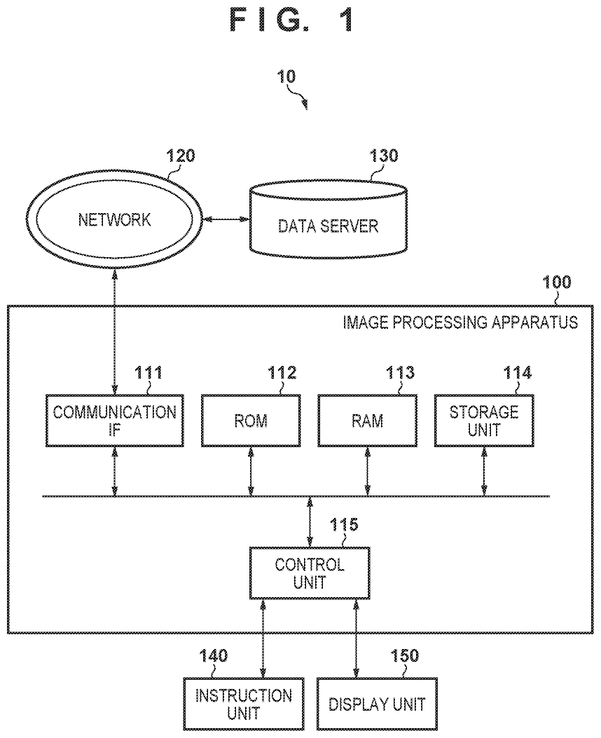

An image processing apparatus according to the first embodiment performs display control of obtaining a projection image in which a plurality of sites of a three-dimensional image are projected at different slab thicknesses, and displaying the obtained projection image on a display unit. By displaying the projection image obtained in the embodiment, the user can observe the plurality of sites projected at suitable slab thicknesses in the projection image, improving the visibility of the plurality of sites in the three-dimensional image. An arrangement and processing according to the embodiment will be described below with reference to FIG. 1.

FIG. 1 is a block diagram showing the arrangement of an image processing system 10 including an image processing apparatus 100 according to the first embodiment. The image processing system 10 includes, as its functional units, the image processing apparatus 100, a network 120, and a data server 130. The image processing apparatus 100 is communicably connected to the data server 130 via the network 120. The network 120 includes, for example, a Local Area Network (LAN) and a Wide Area Network (WAN).

The data server 130 is a Picture Archiving and Communication System (PACS) that holds and manages a medical image and information associated with the medical image. The image processing apparatus 100 can obtain via the network 120 a medical image held in the data server 130. The data server 130 receives and archives an image shot by a medical imaging apparatus (modality), and transmits the image to each apparatus in response to a request from an apparatus connected to the network 120. The data server 130 includes a database that can archive a received image and various data associated with the image.

In the embodiment, an image captured by an X-ray CT apparatus will be exemplified as a three-dimensional image, but another modality may capture a three-dimensional image. Examples of the modality are an Mill apparatus, a SPECT apparatus, and a PET apparatus in addition to the X-ray CT apparatus. The image processing apparatus 100 according to the embodiment is applicable to a three-dimensional image obtained by various modalities.

The image processing apparatus 100 performs image processing according to the embodiment. The image processing apparatus 100 is an apparatus that generates the projection image of a three-dimensional image and displays it on a display unit 150, and functions as an image interpretation terminal apparatus that is operated by a user such as a doctor. The image processing apparatus 100 includes a communication Interface (IF) 111 (communication unit), a Read Only Memory (ROM) 112, a Random Access Memory (RAM) 113, a storage unit 114, and a control unit 115. The image processing apparatus 100 is connected to an instruction unit 140 and the display unit 150.

The communication IF 111 (communication unit) is formed from a LAN card or the like, and implements communication between an external apparatus (for example, the data server 130) and the image processing apparatus 100. The ROM 112 is formed from a nonvolatile memory or the like, and stores various programs. The RAM 113 is formed from a volatile memory or the like, and temporarily stores various types of information as data. The storage unit 114 is formed from a Hard Disk Drive (HDD) or the like, and stores various types of information as data.

The instruction unit 140 is formed from a Graphical User Interface (GUI) including a keyboard, a mouse, and a touch panel, and inputs an instruction from a user (for example, a doctor) to the image processing apparatus 100. An image to be processed is input to the image processing apparatus 100 in accordance with an instruction from the user operating the instruction unit 140. Note that selection of an image may not be based on an instruction from the user, and an image to be processed may be automatically selected by the control unit 115 of the image processing apparatus 100 based on a predetermined rule.

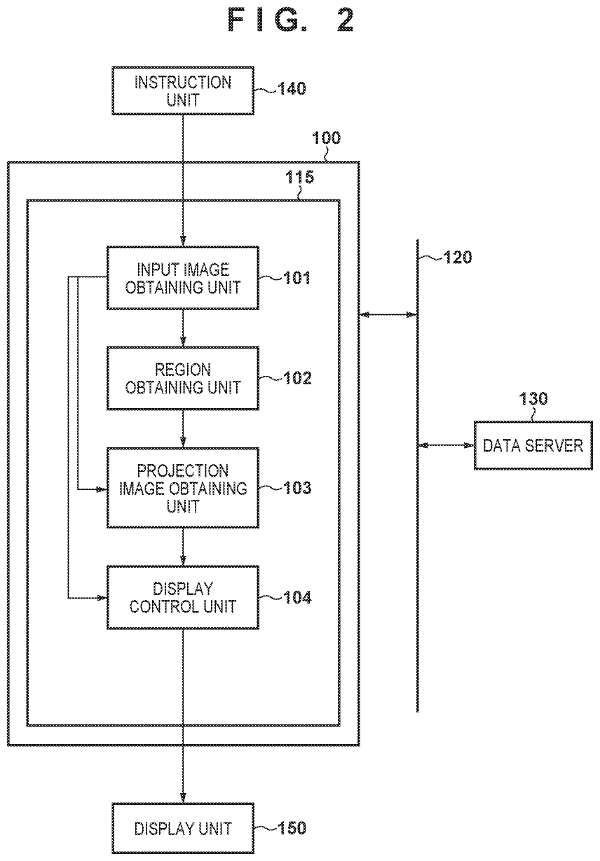

FIG. 2 is a block diagram showing the functional arrangement of the control unit 115. The control unit 115 is formed from a Central Processing Unit (CPU) or the like, and performs overall control of processes in the image processing apparatus 100. The control unit 115 includes, as its functional units, an input image obtaining unit 101, a region obtaining unit 102, a projection image obtaining unit 103, and a display control unit 104.

The input image obtaining unit 101 obtains an input image to be processed from the data server 130 via the communication IF 111 (communication unit) and the network 120. The region obtaining unit 102 obtains the region of an observation object from the input image. The projection image obtaining unit 103 generates a projection image from the input image. The display control unit 104 performs generation of an image to be displayed on the display unit 150 and display control of the generated image.

The display unit 150 is formed from an arbitrary device such as an LCD or a CRT, and displays various types of information such as an image to the user. More specifically, the display unit 150 displays an input image and a projection image obtained from the image processing apparatus 100.

The above-described constituent elements of the image processing apparatus 100 function in accordance with a computer program. For example, the control unit 115 (CPU) loads and executes a computer program stored in the ROM 112 or the storage unit 114 using the RAM 113 as a work area, thereby implementing the functions of the constituent elements. Note that some or all of the constituent elements of the image processing apparatus 100 may be implemented using a dedicated circuit. The functions of some of the constituent elements of the control unit 115 may be implemented using a cloud computer.

For example, the functions of the constituent elements of the image processing apparatus 100 or the control unit 115 may be implemented by communicably connecting via the network 120 the image processing apparatus 100 and an arithmetic apparatus installed at a place different from the image processing apparatus 100, and exchanging data between the image processing apparatus 100 and the arithmetic apparatus.

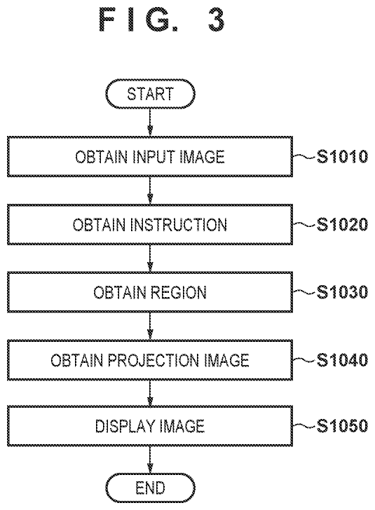

Next, an example of processing of the image processing apparatus 100 in FIG. 1 will be explained with reference to FIG. 3. FIG. 3 is a flowchart showing an example of the processing procedures of the image processing apparatus 100. In the embodiment, processing of obtaining a projection image from a three-dimensional image will be explained by exemplifying a CT image capturing a subject. However, the embodiment is applicable to even an image obtained by another modality.

(S1010: Obtainment of Input Image)

In step S1010, if the user inputs via the instruction unit 140 an instruction to obtain a three-dimensional image, the input image obtaining unit 101 obtains an image (image data) designated by the user as an input image from the data server 130. The input image obtaining unit 101 outputs the obtained input image to the region obtaining unit 102, the projection image obtaining unit 103, and the display control unit 104.

(S1020: Obtainment of Instruction)

In step S1020, the control unit 115 obtains the user instruction via the instruction unit 140. As the user instruction, the site name of an observation object designated by the user, the position of a section of interest, and a slab thickness in projection processing performed on the site of the observation object are obtained. The control unit 115 outputs the obtained site name of the observation object and the obtained position of the section of interest to the region obtaining unit 102, and the position of the section of interest and the slab thickness to the projection image obtaining unit 103.

In the embodiment, the user instruction may be an instruction other than the site name of an observation object, the position of a section of interest, and a slab thickness. For example, the user instruction may be a general known operation (for example, scaling of an image or luminance conversion) performed when an image is displayed on the display unit 150 and the user observes the image. Note that a description of the known operation will be omitted in the embodiment. Regardless of the user instruction, the site name of a predetermined arbitrary observation object, the position of a section of interest, and a slab thickness may be obtained from, for example, the data server 130 or the storage unit 114.

The site name of an observation object may be selectable by the user via the instruction unit 140 from a plurality of observation object candidates (for example, lung, heart, and bone) displayed on the display unit 150.

(S1030: Obtainment of Region)

In step S1030, the region obtaining unit 102 obtains the first region in a tomographic image included in the three-dimensional image, and a second region different from the first region. For example, the region obtaining unit 102 obtains the first region which includes the lung or the heart in the tomographic image included in the three-dimensional image, and the second region different from the first region. Based on the site name of the observation object obtained in step S1020, the region obtaining unit 102 obtains the region of the observation object in the input image. The region obtaining unit 102 outputs the obtained regions of the observation object to the projection image obtaining unit 103. The region obtaining unit 102 obtains the regions of the observation object as a mask image in which a pixel value (for example, 255) representing the site of the observation object is stored at pixels of the region of the observation object, and a different pixel value (for example, 0) is stored at pixels other than the region of the observation object. A predetermined region of the observation object may be a three-dimensional region of the site of the observation object included in the input image, or a two-dimensional region on the section of interest obtained in step S1020.

Although processing will be explained using the lung as an example of the site of the observation object in the embodiment, the embodiment is also applicable to another site. For example, the site may be an arbitrary organ such as the heart or the liver, or the blood vessel, the windpipe, or the bone. The number of sites of the observation object is not limited to one, and it is also possible to obtain the site names of a plurality of observation objects in step S1020 and obtain the regions of the respective sites in step S1030.

The region of the lung can be obtained using a known image processing technique. For example, the processing may be arbitrary threshold processing using the threshold of a pixel value with respect to an input image, or known segmentation processing such as graph cut processing. Alternatively, data (mask image of the lung) representing region information of the lung may be obtained from the data server 130. It is also possible to display an arbitrary two-dimensional tomographic image (section of interest) in an input image on the display unit 150 based on a user instruction or the like, and set, as the region of the observation object, an arbitrary region designated by the user via the instruction unit 140 in the displayed two-dimensional tomographic image.

(S1040: Obtainment of Projection Image)

In step S1040, the projection image obtaining unit 103 obtains a projection image generated by performing projection processing on the input image obtained in step S1010. The projection image is an image generated by setting a “slab” having a predetermined thickness (slab thickness) for each region on the section of interest in the image space, and projecting pixels in the slab onto the section of interest. In other words, the projection image is an image in which the pixel values of respective pixels at corresponding positions in the images of several slices preceding and succeeding the section of interest are collected (projected) on the section of interest.

The section of interest is a section at a position designated by the user via the instruction unit 140. Note that the section of interest is not limited to the position designated by the user, and may be a predetermined arbitrary section such as a slice (axial plane) of the upper or lower end of an input image in the axial direction or a slice at the center. The section of interest is not limited to the axial plane, and may be a section in an arbitrary direction such as the coronal plane or the sagittal plane. The number of sections of interest is not limited to one, and a plurality of sections may be designated to generate a projection image of each section. Alternatively, projection images corresponding to the axial plane, coronal plane, and sagittal plane of an input image may be generated.

The projection image obtaining unit 103 performs projection processing based on an adaptively determined slab thickness with respect to the position of a pixel (pixel of interest) to be processed on the section of interest. The adaptively determined slab thickness is a slab thickness set in accordance with a site to which the pixel of interest belongs. A site to which the pixel of interest belongs can be identified by whether the position of the pixel of interest falls within the region obtained by the region obtaining unit 102 in step S1030. The projection image obtaining unit 103 sets a slab having different slab thicknesses between the inside and outside of a predetermined region in the tomographic image included in the three-dimensional image, and generates a projection image regarding the tomographic image using the set slab. The projection image obtaining unit 103 sets different slab thicknesses for respective regions including different sites in the tomographic image. The projection image obtaining unit 103 sets the first slab having the first slab thickness for the first region in the tomographic image, and the second slab having the second slab thickness for the second region different from the first region. The projection image obtaining unit 103 generates a projection image regarding the tomographic image using the first and second slabs.

The projection image obtaining unit 103 sets a slab thickness based on the form of a site included in a predetermined region. The projection image obtaining unit 103 sets the first slab thickness inside the first region including the first site (for example, lung), and the second slab thickness different from the first slab thickness inside the second region including the second site (for example, heart) different from the first site. For example, when the pixel of interest is a pixel in the region (inside the region) of the lung, the projection image obtaining unit 103 sets a slab thickness (for example, 5 mm) suitable for observation of the form of the site such as the structure of a local blood vessel or bronchus in the lung field. When the pixel of interest is a pixel in the region (inside the region) of the heart, the projection image obtaining unit 103 sets a slab thickness (for example, 10 mm) suitable for observation of the form of the site such as the attribute of the heart wall. When the pixel of interest is neither a pixel in the region of the lung nor a pixel in the region of the heart, the projection image obtaining unit 103 sets a slab thickness (for example, 1 mm) suitable for grasp of the anatomical position of the section of interest.

Although an example in which a projection image is obtained by the MIP method will be explained as an example of projection processing in the embodiment, another projection processing is also applicable in the embodiment. For example, projection processing may be arbitrary one such as the Minimum Intensity Projection (MinIP) method or the average intensity projection method. Projection processing may be a predetermined method or designated by the user via the instruction unit 140. Alternatively, projection processing may be changed in accordance with the site of an observation object.

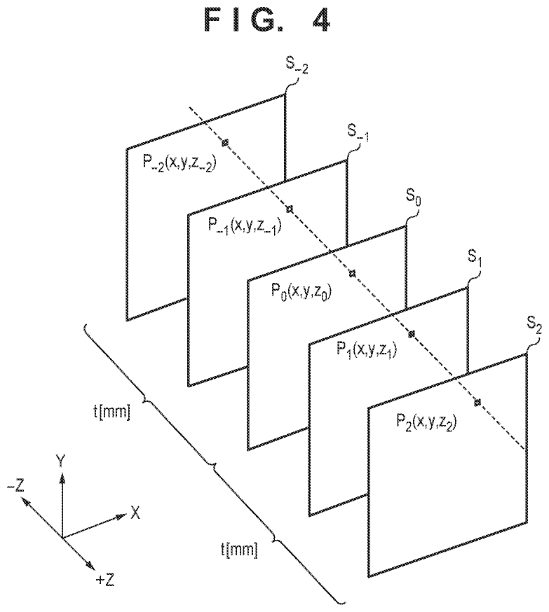

FIG. 4 is a view for schematically explaining projection processing, and shows an example in which the slab thickness of a region to which a pixel P₀ of interest belongs is set to be 2t [mm] (=section of interest+two preceding slices and two succeeding slices). The slab thickness is a numerical value representing a distance from the section of interest (tomographic image). In FIG. 4, the pixel P₀ of interest is a pixel on a section S₀ of interest, and P₀(x, y, z₀) is a pixel value at coordinates (x, y, z₀). In a slice S₁ in the +Z direction, a pixel value at a position corresponding to the pixel P₀ of interest on the section S₀ of interest is represented by P₁(x, y, z₁). In a slice S₂, a pixel value at a position corresponding to the pixel P₀ of interest on the section S₀ of interest is represented by P₂(x, y, z₂). This also applies to the −Z direction. In a slice S₋₁, a pixel value at a position corresponding to the pixel P₀ of interest on the section S₀ of interest is represented by P₋₁(x, y, z₋₁). In a slice S₋₂, a pixel value at a position corresponding to the pixel P₀ of interest on the section S₀ of interest is represented by P₋₂(x, y, z₋₂).

As shown in FIG. 4, when projection processing is performed on the pixel P₀ of interest and pixels (=2×2) of two slices preceding and two slices succeeding the pixel of interest, the pixel values of respective pixels at corresponding positions in the slice images are collected (projected) on the section of interest. For example, in the example of FIG. 4, the pixel value of a projection image can be obtained by collecting (projecting), on the section S₀ of interest, the pixel values of respective pixels P₋₂, P₋₁, P₀, P₁, and P₂ at corresponding positions in the images of two slices preceding and two slices succeeding the section S₀ of interest. That is, in the embodiment, the projection image is generated by obtaining a maximum pixel value of five pixels based on a slab thickness set for each pixel of interest on the section of interest in an input image, and projecting the maximum pixel value at the position of the pixel of interest in the projection image. The method of collecting (projecting), on a section of interest, the pixel values of pixels of the section S₀ of interest and preceding and succeeding slices in FIG. 4 is merely an example. For example, a maximum pixel value may be obtained within a slab centered at the end P₂ in the +Z direction or the end P₋₂ in the −Z direction and projected on the section S₀ of interest.

When a section of interest is a section in an arbitrary direction different from the coordinate axes of an image, projection processing can be performed by collecting (projecting), on the section of interest, the pixel values of pixels corresponding to a pixel of interest in preceding and succeeding slices at positions where the perpendicular of the section of interest passing through the pixel of interest and the preceding and succeeding slices cross each other.

A maximum pixel value is obtained from all pixels included in a set slab in FIG. 4, but projection processing may be performed using only pixels of a region to which a pixel of interest belongs, out of pixels included in the slab. For example, a projection image may be generated based on pixels included in a site in the slab. More specifically, a region to which the pixel P₀ of interest and P₁ and P₂ belong in FIG. 4 is the region of the lung of the observation object. When a region to which P₋₁ and P₋₂ belong is not the region of the observation object, a maximum pixel value of P₀, P₁, and P₂ is set as the pixel value of the pixel of interest in the projection image. This can prevent degradation of the visibility of the site of the observation object caused by projecting, in the region of the observation object on the section of interest, the pixel value of a region other than the site of the observation object.

Alternatively, even if a pixel included in a slab belongs to a region to which a pixel of interest belongs, a pixel determined as noise may not be used for projection processing. For example, for a CT image, a pixel not included in the CT value (for example, −1000 to 100) of a tissue in the region of a general lung can be determined as noise. This can prevent degradation of the visibility of the site of the observation object caused by projecting noise in the region of the observation object on the section of interest.

As the slab thickness, an arbitrary value designated by the user via the instruction unit 140 in step S1020 can be used. Alternatively, a slab thickness determined in advance for the site of an observation object may be obtained from the storage unit 114 or the data server 130. Alternatively, a slab thickness may be set in accordance with the image feature amount of a pixel of interest and surrounding pixels. For example, a slab thickness may be set based on feature information (to be also referred to as an image feature amount hereinafter) of a site obtained from image information (for example, pixel value) of a pixel included in the site of an observation object. Alternatively, a slab thickness may be set based on a comparison between the image feature amount and the threshold. For example, when the site of the observation object is the blood vessel, an image feature amount representing the likelihood of a line structure may be calculated by a known image processing technique, and when the image feature amount of a pixel of interest and surrounding pixels is equal to or larger than the threshold, the pixel may be regarded as one representing the blood vessel, and a slab thickness suited to observe the blood vessel may be set. Alternatively, the variance of the pixel values of a pixel of interest and surrounding pixels may be used as the image feature amount. For example, a region in which pixel values are almost equal and the variance is small may be regarded as a region representing a large structure such as the fat or the muscle, a region having a large variance may be regarded as a region representing a fine structure such as the blood vessel, and slab thicknesses suited to the respective regions may be set. In this case, the processing of obtaining the region of an observation object in step S1030 can be omitted. The image feature amount is not limited to a feature amount representing the likelihood of a line structure, and another image feature amount that can be obtained by a known image processing technique is also applicable.

A slab thickness may be set for a pixel included in the region of an observation object by the above-described method, and a predetermined fixed value (for example, 1 mm) may be set as the slab thickness of a pixel not included in the region of the observation object. The slab thickness of a pixel not included in the region of the observation object may be set to be 0 mm, and the MIP method may not be applied to a pixel having the slab thickness of 0 mm. Alternatively, a slab thickness may be set for only a pixel included in the region of the observation object, and no slab thickness may be set for a pixel not included in the region of the observation object. In other words, the MIP method may not be applied to a pixel not included in the region of the observation object. For example, when the observation object is the lung and the heart, a slab thickness (for example, 5 mm) suited to observe the structures of a local blood vessel and bronchus is set for the region of the lung, a slab thickness (for example, 10 mm) suited to observe the attribute of the heart wall is set for the heart, and projection processing is performed. Then, a projection image is generated in which the pixel value of a section of interest in an input image is stored in a region other than the observation object without performing projection processing. In this case, the lung and the heart can be simultaneously observed in one projection image at their suitable slab thicknesses, and the respective structures can be visually checked efficiently. Generally in a projection image, it is sometimes difficult to grasp position information in the direction of depth. To solve this, the pixels of a section of interest in an input image are displayed without projecting a region (for example, rib) around the site of an observation object, and the projected region of the observation object and the surrounding region are compared. While confirming position information, the structure of the observation object can be visually checked.

(S1050: Display of Image)

In step S1050, the display control unit 104 performs control of obtaining the input image and the projection image from the input image obtaining unit 101 and the projection image obtaining unit 103, and displaying them on the display unit 150. The display control unit 104 also performs control of generating an image to be displayed on the display unit 150 based on the images obtained from the input image obtaining unit 101 and the projection image obtaining unit 103, and displaying the generated image on the display unit 150.

The display control unit 104 has the functions of a general medical image viewer, and has a function (slice display function) of obtaining (cutting out) a slice image (section of interest) as an arbitrary two-dimensional tomographic image in an input image in accordance with a user instruction via the instruction unit 140, and displaying the obtained slice image on the display unit 150. The display control unit 104 also has a function of displaying, on the display unit 150, the projection image of the section of interest (the same section position as that of the slice image of the input image) obtained in step S1030.

The display control unit 104 has a switching display mode in which the slice image of an input image and a projection image are switched in accordance with a user instruction via the instruction unit 140, and either image is displayed on the display unit 150. Alternatively, the projection image may always be displayed. In this case, obtainment of the slice image of an input image can be omitted. The display control unit 104 may have an interlocking display mode in which the slice image of an input image and a projection image are displayed side by side on the display unit 150, and positions of a section of interest in the two images are simultaneously changed and displayed in accordance with a user instruction.

Details of a series of processes in steps S1020 to S1050 have been described above, and the processes in steps S1020 to S1050 can be repetitively executed in the embodiment. That is, a user instruction can be newly obtained via the instruction unit 140 in step S1020 to repetitively execute the processes in steps S1030 to S1050 in accordance with the newly obtained instruction.

For example, the region of an observation object may be changed in accordance with a user instruction via the instruction unit 140. That is, the region of a newly designated observation object is obtained in step S1030 in accordance with a user instruction via the instruction unit 140, and the display control unit 104 performs control of displaying a projection image newly obtained in step S1040 on the display unit 150. When the region of the observation object designated by the user has already been obtained, step S1030 may be skipped. For example, when the region of the observation object designated by the user is obtained and output to the storage unit 114 in step S1030, and the user designates again the site name of the same observation object, the projection image obtaining unit 103 may obtain in step S1040 a projection image using the region of the observation object obtained from the storage unit 114. Then, the display control unit 104 performs control of displaying the obtained projection image on the display unit 150.

The section of interest may be changed in accordance with a user instruction via the instruction unit 140. That is, the projection image of a newly designated section of interest is obtained in step S1040 in accordance with a user instruction via the instruction unit 140, and the display control unit 104 performs control of displaying the new projection image obtained in step S1040 on the display unit 150. When the projection image has already been obtained for the section of interest designated by the user, step S1040 may be skipped. For example, the projection image obtained for the section of interest designated by the user in step S1040 may be output to the storage unit 114, and when the user designates again the same section of interest, the display control unit 104 may perform control of obtaining the projection image from the storage unit 114 and displaying it on the display unit 150.

A slab thickness in projection processing performed on the site of an observation object may be changed in accordance with a user instruction via the instruction unit 140. That is, a projection image of a new slab thickness is obtained in step S1040 in accordance with a user instruction via the instruction unit 140, and the display control unit 104 performs control of displaying the new projection image obtained in step S1040 on the display unit 150. When the projection image has already been obtained for the slab thickness designated by the user, step S1040 may be skipped. For example, the projection image obtained at the slab thickness designated by the user in step S1040 may be output to the storage unit 114, and when the user designates again the same slab thickness, the display control unit 104 may perform control of obtaining the projection image from the storage unit 114 and displaying it on the display unit 150.

Note that the display control unit 104 may archive a generated projection image in the storage unit 114 of the image processing apparatus 100 or the data server 130 via the network 120 in the processing of step S1040. The projection image can be displayed on another arbitrary medical image viewer. In this case, the image display in step S1050 need not always be performed.

According to the first embodiment, the visibility of a local structure and position information of the site of an observation object in a three-dimensional image can be improved by generating a projection image at a slab thickness corresponding to the site of the observation object.

(Modification 1-1)

As for the slab thickness of an observation object set when obtaining a projection image in step S1040, different slab thicknesses may be set in accordance with positions in a site included in the region of the observation object. The slab thickness may be changed in accordance with the position of a pixel of interest with respect to the region of an observation object. For example, when the observation object is the lung, the structures of the blood vessel and bronchus become larger in a region closer to the mediastinum and smaller in a region closer to the thorax. Hence, a projection image may be generated by increasing the slab thickness for a region on the mediastinal side and decreasing it for a region on the thoracic side in accordance with the size of the structure of the lung of the observation object. In this case, projection processing can be performed more efficiently than projection processing performed on the entire lung at a large slab thickness corresponding to the structure on the mediastinal side. By using a proper slab thickness in accordance with the position of a pixel of interest in the region of the observation object, mixing of noise and a pixel value in a region other than the observation object can be prevented, and the visibility of the site of the observation object can be improved.

(Modification 1-2)

In step S1030, the region obtaining unit 102 obtains the region of an observation object as a mask image, but the image may be an image having different pixel values even in the region of the same observation object.

For example, the image may be a likelihood map representing a probability at which the site exists inside the region of the observation object. In this case, the slab thickness of the region of the observation object may be set for each pixel in accordance with the likelihood. For example, a value proportional to the likelihood may be the slab thickness=reference slab thickness×likelihood (0 to 1). The reference slab thickness represents a slab thickness set in the embodiment. When the likelihood of a pixel of interest is equal to or lower than the threshold (for example, 0.1), the pixel of interest is highly likely not to be the observation object, and no projection processing may be performed. Hence, the slab thickness becomes small at a pixel having a low likelihood (for example, at the end of the region of the observation object), reducing mixing of a pixel value in a region other than the observation object by projection processing.

(Modification 1-3)

Processing has been described by targeting a medical image in the embodiment, but may be applied to a three-dimensional image other than a medical image. For example, the processing may be applied to an industrial three-dimensional image capturing an industrial member. The modality may be a CT apparatus or another one. In this case, a projection image can be generated by performing projection processing on, as an observation object, a component inside an industrial member and performing no projection processing on the outline of the industrial member other than the component of the observation object. For example, while the form of a defect (crack, bubble, or foreign substance) of the internal component projected on the projection image is visually checked, the positional relationship with the surroundings can be easily grasped using the outline not projected.

(Modification 1-4)

In the processing of step S1020, the site name of an observation object is obtained in accordance with a user instruction, but a predetermined site name may not always be obtained. For example, an arbitrary region designated by the user via the instruction unit 140 in step S1030 may be set as the region of an observation object without obtaining the site name of the observation object in step S1020. In this case, the site name of the observation object is not obtained, so the projection image obtaining unit 103 performs projection processing based on not a slab thickness corresponding to the site, but a predetermined slab thickness or a slab thickness corresponding to an image feature amount. Alternatively, no projection processing is performed except for the region of the observation object, and a projection image in which the pixel value of a section of interest in an input image is stored is obtained and displayed. Only a local region the user wants to observe can be projected and displayed, and processing can be performed efficiently.

### Second Embodiment

In the first embodiment, a projection image generated from one three-dimensional image is displayed on the display unit. In the second embodiment, a moving image formed from a plurality of projection images generated from a three-dimensional moving image including a plurality of three-dimensional images (frames) is displayed on the display unit so that dynamic information of the observation object can be visually checked easily.

The arrangement of an image processing system 10 according to the second embodiment is similar to that in the first embodiment described with reference to FIG. 1. A flowchart showing the procedures of overall processing performed by an image processing apparatus 100 in the second embodiment is similar to that in the first embodiment described with reference to FIG. 3. In the following description, only a difference from the first embodiment will be explained.

In the second embodiment, processing of obtaining a plurality of projection images from a three-dimensional moving image will be described by exemplifying the moving image of a CT image capturing a subject. However, the embodiment is also applicable to a moving image obtained by another modality.

(S1010: Obtainment of Input Image)

In step S1010, if the user inputs via an instruction unit 140 an instruction to obtain a three-dimensional moving image, an input image obtaining unit 101 obtains a moving image (moving image data) designated by the user as an input image from a data server 130. The input image obtaining unit 101 outputs the obtained input image to a region obtaining unit 102, a projection image obtaining unit 103, and a display control unit 104.

(S1020: Obtainment of Instruction)

In step S1020, similar to the first embodiment, a control unit 115 obtains, via the instruction unit 140, the site name of an observation object designated by the user, the position of a section of interest, and a slab thickness in projection processing performed on the site of the observation object. The control unit 115 outputs the obtained site name of the observation object and the obtained position of the section of interest to the region obtaining unit 102, and the position of the section of interest and the slab thickness to the projection image obtaining unit 103.

In the embodiment, the frame number of the moving image displayed on the display unit 150 may be designated by the user and output to the display control unit 104. If there is no user instruction, a predetermined frame number may be output.

(S1030: Obtainment of Region)

In step S1030, similar to the first embodiment, the region obtaining unit 102 obtains a region in the input image of the observation object based on the site name of the observation object obtained in step S1020. The region obtaining unit 102 outputs the obtained region of the observation object to the projection image obtaining unit 103.

In the embodiment, the three-dimensional region of the site of the observation object included in each frame of the moving image may be obtained, or the two-dimensional region of the site of the observation object at the same section position (section of interest) in each frame may be obtained. The region of the observation object may be obtained from only the section of interest in the frame of the frame number obtained in step S1020.

(S1040: Obtainment of Projection Image)

In step S1040, the projection image obtaining unit 103 obtains a projection image generated by performing projection processing on the input image obtained in step S1010.

In the second embodiment, a projection image may be obtained from each frame of the moving image by a method similar to that in the first embodiment, or a projection image may be obtained from the frame of the frame number obtained in step S1020.

In the embodiment, the projection image obtaining unit 103 obtains a projection image using a slab thickness corresponding to dynamic information of the site of the observation object. For example, when the site name of the observation object is the lung, the projection image obtaining unit 103 generates a projection image at a slab thickness corresponding to the movement of the lung caused by breathing. In this case, even when a position corresponding to an anatomical position (for example, the position of the branch of the bronchus) on a section of interest in a predetermined frame does not exist on the section of interest in another frame, it can be projected on the section of interest in the other frame by projection processing as long as it is included in the slab. That is, even when the user switches a frame (plays back a moving image) while observing a section of interest in a predetermined frame of a three-dimensional moving image, he/she can visually check the structure of the observation object without losing the anatomical position. Note that the dynamic information of the observation object can be obtained based on a result of processing by a known method such as image alignment processing between frames of an input image. At this time, the inside and outside of the region of the observation object can be individually aligned to obtain their pieces of dynamic information. A slab thickness corresponding to the moving amount of movement of the site of the observation object between successive frames may be set for each frame, or the typical value (for example, a maximum moving amount, a minimum moving amount, or an average moving amount) of the moving amount between all frames may be set as a slab thickness common to all the frames. Note that the slab thickness may be set by properly combining dynamic information and the slab thickness setting method described in the first embodiment or the modifications.

(S1050: Display of Image)

In step S1050, the display control unit 104 performs display control similar to that in the first embodiment. Since the input image is a moving image in the second embodiment, a frame can be switched and displayed in accordance with a user instruction.

According to the second embodiment, the moving image of a projection image at a slab thickness corresponding to an observation object can be played back, improving the visibility of dynamic information of the site of the observation object in the three-dimensional moving image.

The present invention is not limited to the above embodiments and various changes and modifications can be made within the spirit and scope of the present invention. Therefore, to apprise the public of the scope of the present invention, the following claims are made.

### Other Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™), a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2022-037244, filed Mar. 10, 2022, which is hereby incorporated by reference herein in its entirety.

What is claimed is:

1. An image processing apparatus comprising:
a region obtaining unit configured to obtain a first region in a tomographic image included in a three-dimensional image, and a second region different from the first region;
a setting unit configured to set a first slab having a first slab thickness for the first region, and a second slab having a second slab thickness, which is different from the first slab thickness, for the second region; and
a generation unit configured to generate a projection image regarding the tomographic image using the first slab and the second slab.

2. The apparatus according to claim 1, wherein the first slab thickness representing a first distance from the tomographic image is different from the second slab thickness representing a second distance from the tomographic image.

3. The apparatus according to claim 1, wherein the setting unit sets different slab thicknesses for respective regions including different sites in the tomographic image.

4. The apparatus according to claim 1, wherein the setting unit sets at least one of the first slab thickness and the second slab thickness based on a form of a site included in a region.

5. The apparatus according to claim 1, wherein the setting unit sets a different slab thickness in accordance with a position in a site included in a region.

6. The apparatus according to claim 1, wherein the setting unit sets at least one of the first slab thickness and the second slab thickness in accordance with a likelihood at which a site exists inside a region.

7. The apparatus according to claim 1, wherein the setting unit sets at least one of the first slab thickness and the second slab thickness based on feature information of a site obtained from image information of the site included in a region.

8. The apparatus according to claim 1, wherein the setting unit sets at least one of the first slab thickness and the second slab thickness based on a variance of image information obtained from the image information of a site included in a region.

9. The apparatus according to claim 1, wherein the setting unit sets the first slab thickness for the first region including a first site, and the second slab thickness for the second region including a second site different from the first site.

10. The apparatus according to claim 1, wherein the generation unit generates the projection image based on a pixel in a site included in a slab.

11. The apparatus according to claim 10, wherein the generation unit performs projection processing on the first region and does not perform projection processing on the second region.

12. The apparatus according to claim 1, further comprising an image obtaining unit configured to obtain a three-dimensional image,
wherein the image obtaining unit obtains a frame included in a three-dimensional moving image as the three-dimensional image.

13. The apparatus according to claim 1, wherein the setting unit sets at least one of the first slab thickness and the second slab thickness based on dynamic information representing movement of a site included in a frame of a three-dimensional moving image obtained as the three-dimensional image.

14. The apparatus according to claim 1, further comprising a display control unit configured to display the projection image on a display unit.

15. An image processing apparatus comprising:
a region obtaining unit configured to obtain a first region including one of a lung and a heart in a tomographic image included in a three-dimensional image, and a second region different from the first region;
a setting unit configured to set a first slab having a first slab thickness for the first region, and a second slab having a second slab thickness, which is different from the first slab thickness, for the second region; and
a generation unit configured to generate a projection image regarding the tomographic image using the first slab and the second slab.

16. An image processing method for an image processing apparatus, comprising:
obtaining a first region in a tomographic image included in a three-dimensional image, and a second region different from the first region;
setting a first slab having a first slab thickness for the first region, and a second slab having a second slab thickness, which is different from the first slab thickness, for the second region; and
generating a projection image regarding the tomographic image using the first slab and the second slab.

17. An image processing method for an image processing apparatus, comprising:
obtaining a first region including one of a lung and a heart in a tomographic image included in a three-dimensional image, and a second region different from the first region;
setting a first slab having a first slab thickness for the first region, and a second slab having a second slab thickness, which is different from the first slab thickness, for the second region; and
generating a projection image regarding the tomographic image using the first slab and the second slab.

18. A non-transitory computer readable storage medium storing a program for causing a computer to execute each step of an image processing method defined in claim 16.
